(19) United States
(12) Patent Application Publication
(10) Pub. No.: US 2012/0316562 A1
(43) Pub. Date: Dec. 13, 2012

(54) VARIABLE-ANGLE MODULAR SYNTHESIS PLATE AND TOOL FOR THE APPLICATION OF THE PLATE

(76) Inventor: Piero Costa, Monta (CN) (IT)

(21) Appl. No.: 13/522,139

(22) PCT Filed: Jan. 14, 2011
(86) PCT No.: PCT/IB2011/050173

§ 371 Date: Aug. 21, 2012

(30) Foreign Application Priority Data
Jan. 15, 2010 (IT) TO2010A000023

(51) Int. Cl. A61B 17/80; A61B 17/88

(52) U.S. Cl. 606/71; 606/86 B; 606/281

(57) ABSTRACT

The present invention regards a synthesis plate (1), particularly for pelvic osteotomy; the present invention also regards an instrument (101) and a method for the application of the same. According to the invention, said plate (1) is composed of two distinct, separate modular components (3,5) that can be connected and fixed to each other, such that it is possible to adjust the relative angle (CC) between said two components of the modular plate as required, and then fix and integrally join them together in the desired angular position. Advantageously, with the plate (1) according to the invention and the suitable tool (101) for the application thereof, the adjustment and selection of the mutual angular position of said components can be carried out at any time, even during the execution of the osteotomy operation.

### FIELD OF THE ART

The present invention regards a synthesis plate, and more precisely a variable-angle modular synthesis plate, particularly for pelvic osteotomy.

The present invention also regards a tool and method for the application of said plate.

### PRIOR ART

Pelvic osteotomy is a surgical treatment that is known and used, e.g. in the veterinary field, in the treatment of hip dysplasia.

Hip dysplasia is a pathology that is widespread in dogs, especially in large-size dogs (e.g. German shepherds and Molossian dogs). It involves a painful, disabling condition of instability between the acetabulum and the femoral head. Normally, such pathology is of genetic origin, and results from an insufficient formation of the acetabulum.

During the growth of the animal, since the hip is a load-bearing joint, the instability of the mutual position between the acetabulum and the femoral head causes further damage to surrounding tissue.

For the treatment of such pathology, drug therapy is normally not sufficient, and it is necessary to resort to a surgical operation.

Various surgical techniques (together with relative synthesis means) have been proposed in the past, notably pelvic osteotomy. Pelvic osteotomy consists of an osteotomy of the pelvis capable of isolating the acetabular segment and allowing its axial rotation; once the desired degree of rotation has been obtained, the stabilization is executed by means of the application of a synthesis plate.

Operating techniques and synthesis means for obtaining pelvic osteosynthesis for the treatment of hip dysplasia, and of other pathologies in both animals and humans, are described in WO 98/46155 and US 2008/045960, for example.

In particular, U.S. Pat. No. 4,762,122 describes a synthesis plate which allows obtaining the coverage of the femoral head with the acetabulum not by simply rotating the acetabulum itself on its median axis, but by carrying out a rotation-translation of the same on the rotation center of the femoral head. This plate type allows limiting the risk of constriction of the pelvic canal, since the acetabular segment does not medially rotate towards the opposite side of the ilium, but is first offset and then rotated with respect to the center of the femoral head. The synthesis plate described in U.S. Pat. No. 4,762,122 consists of two planar components, tilted with respect to each other by a pre-established angle and connected to each other at their edge by means of an arm, arranged for being situated at a cut made on the ilium during the osteotomy. Even if a synthesis plate of the above-described type has undoubted advantages, it does not however lack drawbacks. Since the tilt angle between the two components of the plate is fixed, it is not possible to adapt the synthesis plate to the anatomy of the patient operated each time, as required, such that the outcome of the fixing is not optimal.

Alternatively, it would be necessary to have a great number of plates, each with the components tilted with respect to each other by a different angle, which would involve high costs and much waste.

In addition, once the synthesis plate has been selected with a specific mutual tilt between the components, it is not possible to modify such tilt in case of need, e.g. during the osteotomy operation.

The object of the present invention is to overcome the aforesaid drawbacks by providing a synthesis plate, with relative application tool and method, which allow varying the tilt between the components of the plate, for the purpose of ensuring optimal coverage of the acetabulum on the femoral head with complete respect for the anatomy surrounding the synthesis means itself.

In particular, the object of the present invention is to provide a synthesis plate which allows varying said relative tilt also during the osteotomy operation.

These and other objects are attained by means of a synthesis plate and a tool and method for the application thereof as claimed in the claim set.

### DESCRIPTION OF THE INVENTION

Due to the fact that the synthesis plate according to the invention has a modular structure and is composed of two components that can be locked to each other but which are free to rotate with respect to each other along a suitably arranged guide, the tilt of said two components can be adjusted according to the requirements of each specific case. Hence, the rotation of the acetabulum on the femoral head is suitably allowed in order to increase the coverage thereof. Thanks to the invention, the adjustment of the relative tilt between the two aforesaid components and the subsequent selection of the optimal angle can be carried out at any time, even during the surgical operation.

Advantageously, the plate according to the invention allows avoiding the problems of constriction of the pelvis canal, preventing breakage or bending of the two parts forming the plate.

Due to the fact that the application tool according to the invention comprises a pair of arms, each of which suitable for being coupled with one of the components of the synthesis plate in order to drive its rotation, it allows—also during surgery—selecting the tilt angle between the two components of the plate which ensures the correct coverage of the acetabulum on the femoral head and which subsequently ensures the possibility of mutual fixing of the two parts of the plate in the appropriate position.

### BRIEF DESCRIPTION OF THE FIGURES

Further characteristics and advantages of the invention will be clearer from the following detailed description of several embodiments thereof, given as a non-limiting example with reference to the enclosed drawings, in which:

FIG. 1 is an exploded perspective view of the synthesis plate according to a first embodiment of the invention;

FIG. 2 is a perspective view of the first component or rear component of the synthesis plate of FIG. 1;

FIG. 3 is a perspective view of the second component or front component of the synthesis plate of FIG. 1;

FIG. 4 is a side view of the synthesis plate of FIG. 1;

FIG. 5 is a perspective view of the first component or rear component of a synthesis plate according to a second embodiment of the invention;

FIG. 6 is a perspective view of the second component or front component of a synthesis plate according to said second embodiment of the invention;

FIG. 7 is a perspective view of the first component or rear component of a synthesis plate according to a third embodiment of the invention;

FIG. 8 is a perspective view of the tool according to the invention for the application of the synthesis plate of FIG. 1.

### DESCRIPTION OF PREFERRED EMBODIMENTS OF THE INVENTION

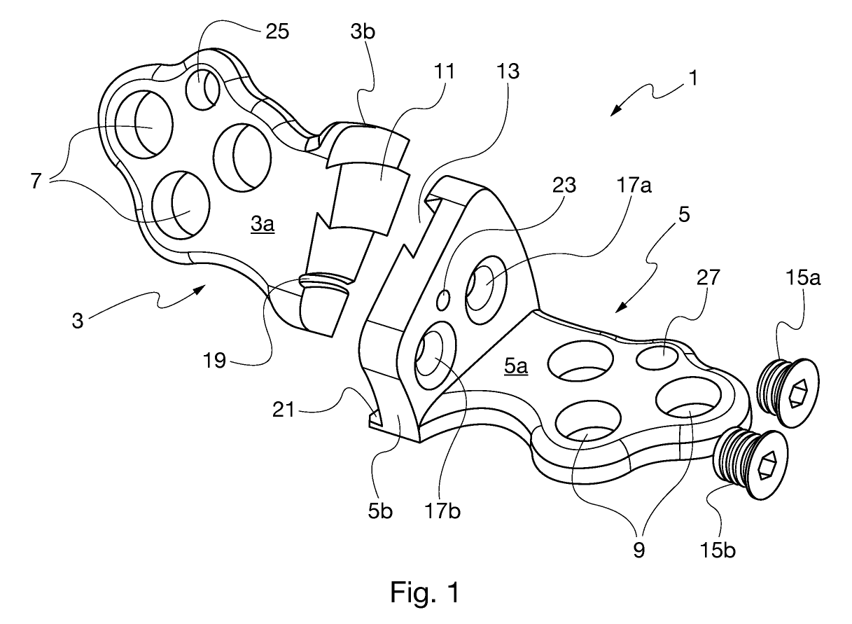

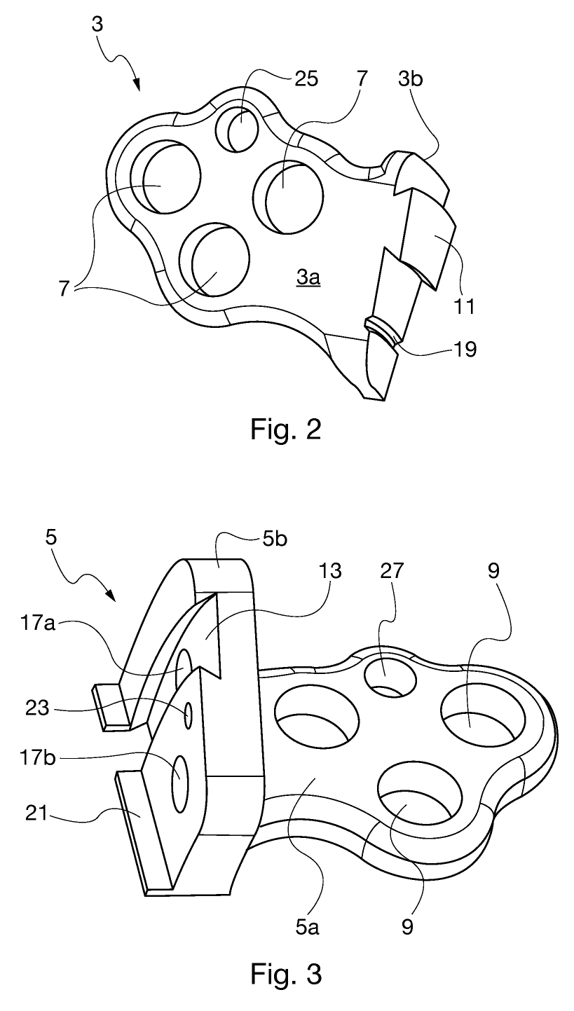

With reference to FIGS. 1-4, a synthesis plate is illustrated, obtained according to a first embodiment of the invention—particularly for use in canine pelvic osteosynthesis on a right ilium section—and indicated in its entirety with the reference number 1.

According to the invention, the synthesis plate comprises a first component or rear component 3 and a second component or front component 5, distinct and separate from each other. Each of said components comprises a fastening portion 3a,5a, respectively, provided with bores 7,9, respectively, for the passage of screws for the fastening to the respective sections of osteotomized bone.

Each of said components also comprises a coupling portion 3b,5b, respectively, arranged on a plane that is substantially perpendicular to the plane where the fastening portion lies. Said coupling portion 3b,5b is provided with coupling means for securing said components 3,5 to each other, in a manner such that they cannot be moved away from each other; nevertheless, such components 3,5 are free to rotate around an axis perpendicular to the plane where said coupling portions 3b,5b lie.

Due to such expedient, the two components 3,5 of the synthesis plate 1 can rotate with respect to each other, such that the angle formed by the planes where the fastening portions 3a,5a of said components lie can be varied as desired.

With specific reference to the coupling means of the embodiment illustrated in FIGS. 1-4, the coupling portion 3b of the first component or rear component 3 is provided with a projection 11 having a curved profile and a dovetail section; correspondingly, the coupling portion 5b of the second component or front component 5 is provided with a recess 13 having a curved profile and a dovetail section. In this manner, the dovetail projection 11 can be inserted in the dovetail recess 13 and retained therein, by locking the two components 3,5 of the synthesis plate 1; on the other hand, the dovetail projection 11 can slide inside the dovetail recess 13, thus allowing the mutual rotation of the components 3,5.

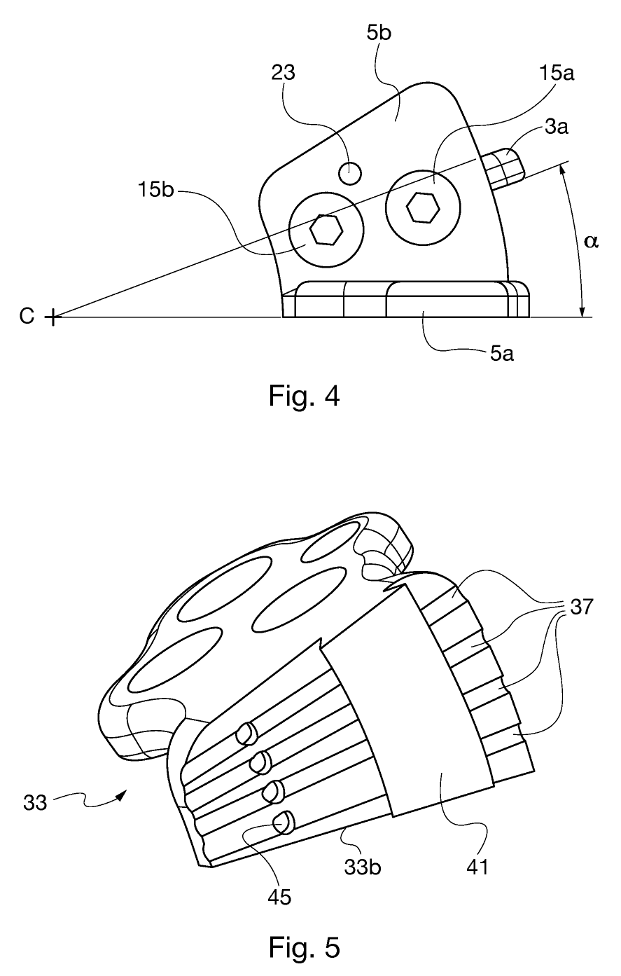

The center of rotation of the curved profile of said projection and said recess can be selected as desired; with particular reference to FIG. 4, said rotation center C preferably lies outside the coupling portions 3b,5b of the components 3,5, and particularly as close as possible to the rotation center of the femur head which is housed by the acetabulum. In this manner, the rotation-translation of the acetabulum during osteotomy is ensured, in accordance with an established technique with consolidated advantages: the two moved ilium parts are indeed rotated and translated, and upon completed rotation the translation allows maintaining the actual initial diameter of the pelvic canal. Therefore, in this manner, the risk of constriction of the pelvic canal is reduced, and the correct anatomy is preserved of the entire posterior locomotor apparatus of the animal.

It is observed that, even if in the illustrated embodiment the first component 3 is provided with a projection 11 and the second component 5 is provided with a corresponding recess 13, it is also possible to provide for a projection on the second component 5 and a corresponding recess on the first component 3. It is also clear that the sections of said projection and said recess are not limited to the dovetail form; any other form that allows the coupling of the two aforesaid components is equally acceptable.

Still with particular reference to FIG. 4, it is clear that the coupling means of the synthesis plate 1 according to the invention allow varying the angle α between the planes of the fastening portions 3a,5a of the components 3, 5: said components can slide along the curved profile of the dovetail coupling which locks them to each other, ensuring a single degree of freedom, the rotation with respect to the rotation center C.

The coupling portions 3b,5b of the components 3,5 are also provided with locking means for rigidly fixing said two components to each other, once the desired mutual tilt angle α has been selected.

With reference to FIG. 1, said locking means are constituted by a pair of screws 15a,15b which penetrate through bores 17a,17b provided for in the coupling portion 5b of the front component 5 of the synthesis plate 1.

Preferably, the bores 17a,17b comprise a first non-threaded conical portion and a second threaded cylindrical portion; correspondingly also the screws 15a,15b comprise a non-threaded conical head and a threaded cylindrical stem. In addition, said screws can comprise known means for preventing the undesired loosening thereof.

A first bore 17a is conveniently provided for at the dovetail recess 13, such that the corresponding screw 15a exerts a pressure on the dovetail projection 11 of the rear component 3 of the plate 1. A second bore 17b is provided for at a curved groove 19 possibly obtained on the coupling portion 3b of the rear component 3 and arranged along an arc that is concentric to that on which the projection 11 extends; correspondingly, the stem of the second screw 15b can terminate with a terminal projection (not illustrated) adapted to be engaged with said groove.

The tightening of the screws 15a,15b, by pushing the walls of the dovetail projection 11 against the walls of the dovetail recess 13, ensures the secure locking of the two components 3,5 of the plate 1 even when the animal places its pelvis under load, once the desired angle α has been selected. In this respect, it is observed that such angle α will generally—but not exclusively—be in the range of 10° to 45°.

In the case of application to pelvic osteotomy, said coupling portions 3b,5b during use lie on a plane which normally coincides with the plane of the cut made on the ilium during pelvic osteotomy, and the components 3,5 of the synthesis plate 1, suitably tilted with respect to each other, are fixed to the respective ilium bone sections.

In this respect, it is observed that the number, the arrangement and the tilt of the bores 7,9 in the fastening portions 3a,5a of said components can be selected as desired, in order to fully respect the anatomy of the pelvis of the animal and to reduce the risk of loosening of the fastening screws, a recurrent risk which causes pain and often requires correction surgery operations.

The geometry itself of the components 3,5 of the synthesis plate 1 can be selected in a manner so as to allow the complete respect of the anatomy of the bones on which the plate is applied; it is therefore possible to provide for particular folds of the plates or relief grooves with thickness variations at the lower parts of the plates in order to ensure easy and secure plate positioning during the pelvic osteotomy operation.

In this respect, it is observed that the size of the surface area of the fastening portions 3a,5a of the components 3,5 represents a substantial portion of the ilium, which must be integrally joined with the use of the plate. In this manner, one ensures optimal resistance to the forces to which the ilium is subsequently stressed.

Still with reference to FIGS. 1-4, the coupling portion 5b of the front component 5 of the plate 1 may be provided with an abutment profile 21 which constitutes an end stop for the dovetail projection 11 and thus limits the mutual rotation of the two components 3,5.

The coupling portion 5a of the front component 5 can also comprise a supplementary bore 23 suitable for the passage of a pin (not illustrated) for maintaining the two components 3,5 locked in a pre-established position, until the adjustment of their mutual tilt is not necessary (e.g. during the transport and storing operations).

Finally, in the fastening portion 3a,5a of each component 3,5 of the synthesis plate 1, a supplementary coupling bore is provided for, 25,27, respectively, whose purpose will be clarified below.

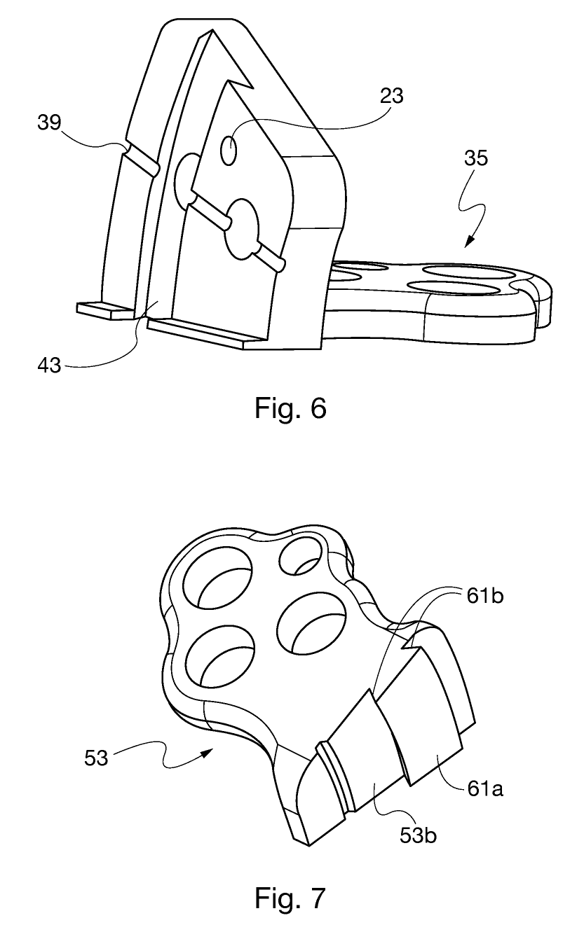

Continuing now to FIGS. 5 and 6, the first component or rear component 33 and the second component or front component are respectively illustrated for a synthesis plate according to a second preferred embodiment of the invention. According to said second embodiment, the adjustment of the mutual angular position of the components of the synthesis plate does not occur in a continuous manner, but rather in a discrete manner.

For such purpose, the coupling portion 33b of the rear component 33 has a plurality of first grooves 37 which allow locking the dovetail projection 41 of the rear component with respect to the dovetail recess 43 of the front component in pre-established positions, corresponding to said grooves. Correspondingly, in the coupling portion 35b of the front component 35, a second groove 39 is obtained in which a wire can be forcibly inserted, such wire of appropriate diameter for blocking any subsequent attempt to rotate the two components with respect to each other.

In addition, in order to optimize the mutual locking of the components 33,35 of the synthesis plate, insertion bores 45 for the terminal portion of a fastening screw can be provided for in the first grooves 37 of the coupling portion 33b of the rear component 33, in the region of one of the bores obtained in the coupling portion 35b of the front component 35.

In FIG. 7, the first component or rear component 53 is illustrated for a synthesis plate according to a third preferred embodiment of the invention.

According to this third embodiment, the dovetail projection 61 of the coupling portion 53b of the rear component 53 has a rough front surface 61a and rough side surfaces 61b, which facilitate the locking of the screw that fixes the mutual angular position of the two components of the plate.

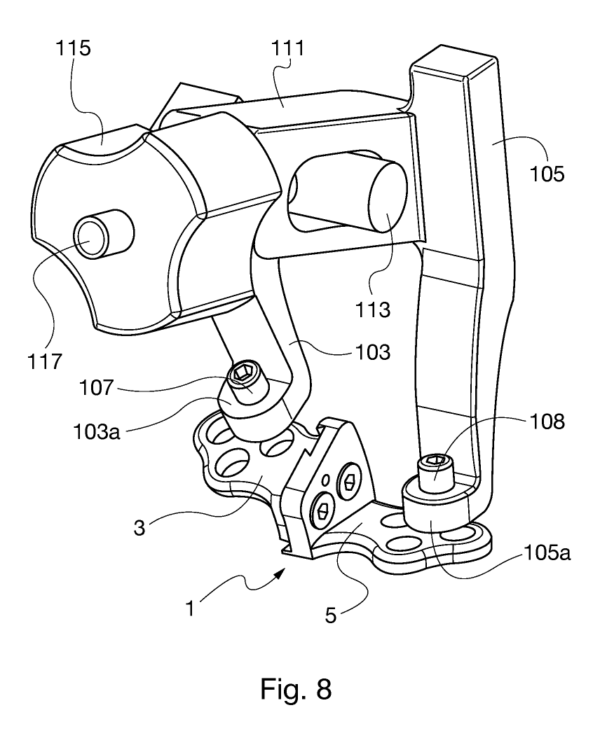

Passing now to FIG. 8, the tool 101 according to the invention is schematically illustrated, such tool being employed for the application of the above-described synthesis plate.

Said tool allows—even during surgery—adjusting the mutual angular position of the two components of the synthesis plate and selecting the desired angle α between them, so as to ensure the coverage of the femoral head by the acetabulum.

The tool 101 comprises a first arm 103 which terminates with a foot 103a provided with a through bore, through which a first screw 107 is made to pass; such screw 107 then penetrates through the coupling bore 25 of the first component 3 in order to fix said first arm to said first component. The tool 101 also comprises a second arm 105 which terminates with a foot 105a provided with a through bore, through which a second screw 109 is made to pass; such screw 109 then penetrates through the coupling bore 27 of the second component 5 in order to fix said second arm to said second component.

The two arms 103,105 of the tool 101 are connected to each other by means of a modular guide 111 which allows a pin 113 to slide in a controlled manner and allows a mutual rotation-translation movement of the two arms 103,105 and of the two components 3,5 of the synthesis plate 1 connected thereto. Such movement is controlled by a ring nut 115 which is screwed/unscrewed on a threaded pin 117, in turn bound to said pin 113.

The operating technique for pelvic osteotomy initially provides for the steps of:
cutting the pubic ramus of the pelvis;
cutting the ischial tuberosity parallel to the sagittal plane, with possible release of the sacrotuberous ligament at its insertion on the ischial tuberosity;
cutting the ilium in the transverse plane, in a posterior position with respect to the sacroiliac joint and in a cranial position with respect to the acetabulum.

At this point, the application method for the synthesis plate according to the invention provides for the steps of:
fixing the tool 101 to the synthesis plate 1 by connecting the arms 103,105 of said tool to the components 3,5 of said plate;
positioning the plate 1 in the section deriving from the cut of the ilium in the transverse plane, possibly locked in minimum tilt position;
fastening, by means of screws, the fastening portions 3a,5a of the components 3,5 of the plate 1 to the respective sections of the ilium;
adjusting the mutual angular position of the components 3,5 of the plate 1 by acting on the driving nut 115 of the tool 101 in a manner so as to rotate said components with respect to each other;
selecting the desired angle α so as to ensure the coverage of the femoral head;
fixing the mutual position of the components 3,5 of the synthesis plate by screwing the screws 15a,15b into the bores 17a,17b;
removing the tool 101 from the synthesis plate 1.

It is observed that the plate according to the invention also allows using an alternative osteotomy technique which provides for removing the pubic ramus of the pelvis and cutting the ilium in the transverse plane, in a position just to the rear of the sacroiliac connection, maintaining the integrity of the ischium.

From that described above, it will be clear that the synthesis plate and the tool and method for applying the same according to the invention attain the objects set forth above, since they allow adjusting the mutual angular position of the two components of the synthesis plate and selecting the desired relative tilt angle, in a manner so as to ensure the necessary coverage of the femoral head and thus stabilize the coxofemoral joint.

It will also be clear that numerous modifications and variants of the above-described embodiments are possible, without departing from the scope of protection of the invention as defined by the appended claims.

In particular, even if the modular synthesis plate illustrated in the above description is intended for use on a right ilium section, it is clear that an exact mirror image of such plate, i.e. a plate with all the same characteristics, can be obtained and used for application on a left ilium section.

1-10. (canceled)

11. Synthesis plate (1), particularly for pelvic osteotomy, comprising a first component or rear component (3;33;53) and a second component or front component (5;35), each of said components comprising a fastening portion (3a,5a), provided with bores (7,9) for the passage of screws for the fastening to respective bone portions, said first and said second component (3,5;33,35;53) being distinct, separate elements, wherein each of said components comprises a coupling portion (3b,5b;33b,35b), said coupling portions (3b,5b;33b,35b) of said components (3,5;33,35;53) lying on planes that are substantially perpendicular to said fastening portions (3a,5a) of said components and being provided with coupling means (11,13;37,39,41,43;61,61a,61b) for securing said components to each other, so that they cannot be moved away from each other, while allowing at the same time the mutual rotation of said components with respect to each other components around an axis that is perpendicular to the plane where said coupling portions (3b,5b) lie and said coupling portions (3b,5b;33b,35b) being further provided with locking means (15a,15b,17a,17b) for rigidly fixing said two components to each other at a desired mutual angular position; and wherein said coupling means allow a mutual rotation of said.

12. Synthesis plate (1) according to claim 11, wherein said coupling means are designed so as to provide a continuous adjustment of the mutual angular position of said components (3,5;33,35;53).

13. Synthesis plate (1) according to claim 11, wherein said coupling means are designed so as to provide a discrete adjustment of the mutual angular position of said components (3,5;33,35;53).

14. Synthesis plate (1) according to claim 11, wherein the coupling portion of one of said components (3,5;33,35;53) is provided with a projection (11;41;61) having a curved profile and a dovetail section and the coupling portion of the other of said components (5,3;35,33) is correspondingly provided with a recess (13;43) having a curved profile and a dovetail section.

15. Synthesis plate (1) according to claim 14, wherein said dovetail projection (61) has rough surfaces (61a,61b).

16. Synthesis plate (1) according to claim 14, wherein said locking means comprise at least a first bore (17a) at said dovetail groove (13) and a first screw (15a) suitable for penetrating through said first bore and for exerting a pressure on said dovetail projection (11).

17. Synthesis plate (1) according to claim 13, wherein the coupling portion of one of said components (33,35) is provided with a plurality of first grooves (37) and the coupling portion of the other of said components (35,33) is correspondingly provided with a second groove (39), a wire being provided for being forcibly inserted into the cavity defined by one of said first grooves and said second groove.

18. Tool (101) for the application of a synthesis plate (1) according to claim 11, characterized in that it comprises:
a first arm (103) suitable for being rigidly connected to said first component (3) of said plate;
a second arm (105) suitable for being rigidly connected to said second component (5) of said plate;
a modular guide (111) connecting said first and second arms (103,105) and allowing a pin (113) to slide in a controlled manner so as to allow a mutual rotation-translation movement of said two arms (103,105) and of said two components (3,5) of said plate connected thereto;
a driving nut (115) which drives the movement of said pin (113).

19. Method for the application of a synthesis plate (1) according to claim 11, characterized in that it comprises the steps of:
fastening said fastening portions (3a,5a) of said components (3,5;,35;53) of said plate to respective surfaces, for example by means of screws;
adjusting the mutual angular position of said two components (3,5;33,35;53) by rotating them with respect to each other;
selecting a desired mutual angular position of said two components (3,5;33,35;53) with respect to each other;
rigidly fixing said two components (3,5;33,35;53) with respect to each other at said desired mutual angular position.
